(19) United States
(12) Patent Application Publication
(10) Pub. No.: US 2023/0405255 A1
(43) Pub. Date: Dec. 21, 2023

(54) NASAL RESPIRATORY APPARATUS HAVING NASAL DAM

(71) Applicant: Pneuma Therapeutics, Inc., Weare, NH (US)

(72) Inventor: David M. Kane, Windham, NH (US)

(21) Appl. No.: 18/027,474

(22) PCT Filed: Sep. 21, 2021
(86) PCT No.: PCT/US2021/051214

§ 371 Date: Mar. 21, 2023

Related U.S. Application Data
(60) Provisional application No. 63/081,100, filed on Sep. 21, 2020.

(51) Int. Cl. A61M 16/06

(52) U.S. Cl. CPC A61M 16/0666; A61M 2202/02; A61M 16/0622

(57) ABSTRACT

A nasal respiratory apparatus includes a nasal interface, such as a nasal dam, and a removable air chamber assembly. The nasal dam may be a solid form with nares ports channeling air flow to an air chamber of the air chamber assembly via nares ports in the air chamber assembly. The nasal dam may be hollow, with a membrane with nares ports interfacing with the nares of a patient for channeling air flow to the air chamber of the air chamber assembly.

### CROSS REFERENCE TO RELATED APPLICATIONS

This application is claims priority to U.S. Provisional Patent Application Ser. No. 63/081,100, filed Sep. 21, 2020, which application is hereby incorporated by this reference in its entirety to the extent allowed by law.

### FIELD

Embodiments of the present invention relate to oxygenation, ventilation, end tidal carbon dioxide (CO2) sampling during general anesthesia and deep sedation, and specifically to a nasal respiratory platform with various related features.

### BACKGROUND

General anesthesia has historically utilized a full-face mask attached to an anesthesia machine to support providing anesthetic gases and oxygen, as well as ventilating the patient and monitoring exhaled end tidal CO₂ levels. A major issue with using a full-face mask is that the mask must be removed for oral access to place an intubation tube, resulting in an apenic period. Respiratory compromise is a common result from the apenic period for high-risk patients.

Given the trend for more minimally invasive procedures, the use of intravenous deep sedation has grown significantly. Nasal cannula are used providing nasal oxygenation, but do not provide pressurization, sometimes resulting in respiratory compromise if the nasal pharynx becomes blocked.

Accordingly, there is a need for a nasal respiratory platform supporting pressurized nasal oxygenation, ventilation, and expired End Tidal CO₂ sampling by interfacing with and sealing about the nasal base of the nose.

A representative inhalation and exhalation cycle (flow and pressure provided by the ventilator) for a patient-ventilator interface during noninvasive ventilation is shown in FIG. 1. Inspiration of gas into the patient's lungs occurs when the flow rate as measured in L/min is positive while expiration occurs when the flow rate as measured in L/min is negative. Note that in this example, a minimum pressure of nominally 5 CM H2O is maintained in order to provide Positive Expiratory End Pressure (PEEP). PEEP is a therapy provided in order to avoid passive emptying of the lung.

To address the shortcomings of full-face masks and nasal cannula, nasal ventilation masks covering the nose and sealing against the face are becoming popular. nasal ventilation masks support pressurization required to overcome blockage of the nasal pharynx, but obstruct the region near the eyes, easily lose a seal if the mask is tilted or if there is facial hair such as a mustache is present.

A nasal respiratory apparatus according to principles described herein and its various embodiments and combinations of features addresses the major shortcomings of all three of these approaches, supporting pressurized oxygenation, ventilation and end-tidal CO₂ sampling via nasal ventilation system that seals via the nares and nasal vestibule. This results in a more secure seal. The device is much more compact an unobtrusive than either mask approach, allowing for oral and eye access if required.

### BRIEF SUMMARY OF THE DISCLOSURE

Accordingly, the present invention is directed to nasal respiratory apparatus that obviates one or more of the problems due to limitations and disadvantages of the related art.

According to principles described herein, a nasal respiratory assembly includes a nasal interface comprising at least one opening for fluid communication with the nares of a patient and an air chamber assembly comprising an air chamber, a gas supply port, an end tidal sample port and at least one opening in fluid communication with the nasal interface, wherein the nasal interface comprises a pliable material shaped to abut and seal a patient's nasal base such that respiratory gasses pass via the patient's nostrils, the at least one opening, the air chamber, the gas supply port, and the end tidal sample port.

In an aspect of the nasal respiratory assembly, the nasal interface includes a cavity of material having Shore A 5-20 durometers, and a membrane extends over the cavity. The at least one opening extends through the membrane into the cavity. The cavity further includes a second opening in a floor of the cavity and in fluid communication with the air chamber via the at least one opening in fluid communication with the nasal interface, whereby a patient's nasal base interface with the membrane for providing a seal.

The nasal interface may comprise a solid material having a roughly rectangular cross section and Shore A 5-20 durometers, whereby the at least one opening extends from a surface of the nasal interface through the solid material to an opposite surface of the material and aligns with the at least one opening in fluid communication with the nasal interface.

### BRIEF DESCRIPTION OF THE DRAWINGS

The accompanying figures, which are incorporated herein and form part of the specification, illustrate a new nasal respiratory apparatus. Together with the description, the figures further serve to explain the principles of the new nasal respiratory apparatus described herein and thereby enable a person skilled in the pertinent art to make and use the new nasal respiratory apparatus

FIG. 1 illustrates a pressurized nasal ventilator assembly in a respiratory system.

FIG. 2 illustrates a nasal respiratory apparatus with a solid nasal dam.

FIG. 3 illustrates nasal dam compression by a patient nasal base to create a seal.

FIG. 4 illustrates a solid nasal dam stiffness model.

FIG. 5 illustrates a nasal respiratory apparatus with a hollow nasal dam.

FIG. 6 illustrates a nasal respiratory apparatus with a hollow nasal dam.

FIG. 7 illustrates an interface between a patient nasal base and a hollow nasal dam.

FIG. 8 shows detail of an embodiment of a hollow nasal dam according to principles described herein.

FIG. 9 shows detail of an air chamber assembly according to principles described herein.

FIG. 10 illustrates a hollow nasal dam stiffness model.

FIG. 11 illustrates a circular plate for modeling behavior of a hollow dam membrane as described herein.

### DETAILED DESCRIPTION

Reference will now be made in detail to embodiments of the new nasal respiratory apparatus with reference to the accompanying figures. For convenience of explanation, various figures make use of a right-handed X, Y, Z-axis Cartesian Coordinate system reference space, with reference to X-Y, X-Z, and Y-Z planes.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

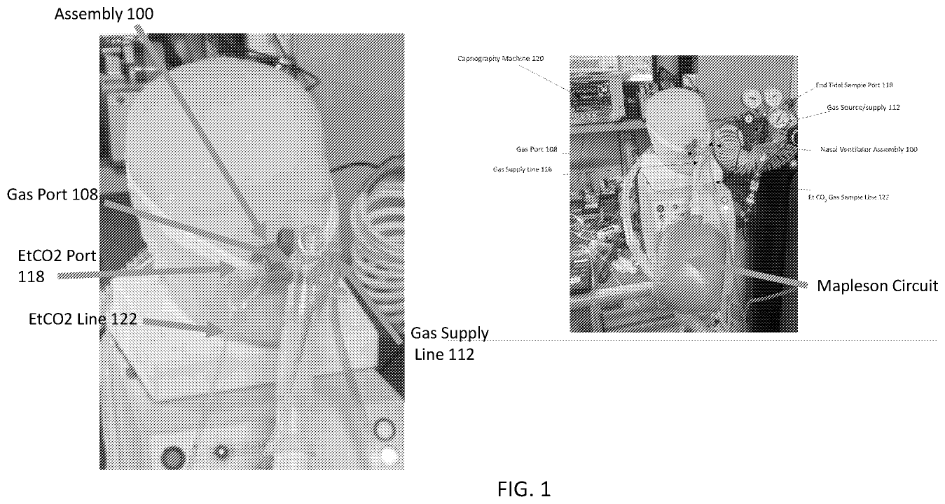

FIG. 1 illustrates an example pressurized nasal ventilator system 100 as applied to a patient according to principles described herein. As shown in FIG. 1, the pressurized nasal ventilator assembly 101 includes an air chamber 106, a gas port 108 connected to a gas supply 112 via a gas supply line 116, and an end tidal sample port 118 connected to a capnography machine 120 via an end tidal gas sample line 122. The pressurized nasal ventilator assembly 100 further includes a “nasal dam” 124 as a substantially sealed interface to the patients nares. While illustrated herein as a nasal dam with a pressurized nasal ventilator assembly, principles described herein are not so limited, and it should be appreciated by those of skill in the art that any nasal interface that provides a substantially sealed interface between the pressurized nasal ventilator assembly 100 and the patient's nares/nostrils could be used in the disclosed pressurized nasal ventilator assembly 100 within the scope of the present disclosure.

FIG. 1 illustrates a nasal dam 124 attached to a nasal ventilator assembly, some features of which were previously disclosed in PCT application no. PCT/US2021/021829, and which claims the benefit of U.S. Provisional Patent Application Ser. No. 62/987,944, filed Mar. 11, 2020, U.S. Provisional Patent Application Ser. No. 62/992,333, filed Mar. 20, 2020, U.S. Provisional Patent Application Ser. No. 63/006,407, filed Apr. 7, 2020 and U.S. Provisional Patent Application Ser. No. 63/006,411, filed Apr. 7, 2020, all pending, which applications are hereby incorporated by this reference in their entirety to the extent allowed by law. The present application is directed to a nasal dam suitable for use for all purposes of all configurations disclosed in PCT/US2021/021829.

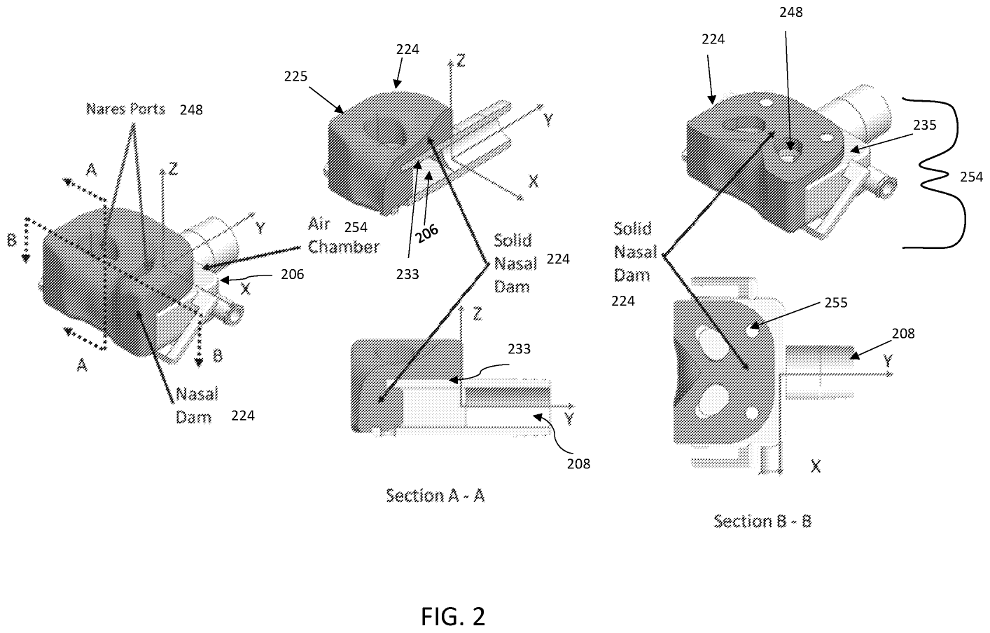

An embodiment of a nasal dam 224 according to principles described herein is illustrated in FIG. 2 in cooperation with an air chamber assembly 254 or/in place of the vent 104 of FIG. 1. FIG. 2. The nasal dam 224 is a solid nasal dam in cooperation with an air chamber 206, as illustrated in FIG. 2. The nasal dam includes a profile to conform to a patient's nasal base at its top surface 225. The nasal dam 224 includes a pair of nares ports 248 therethrough from the top surface 225 of the nasal dam to a bottom surface 233 of the nasal dam, where the bottom surface 227 of the nasal dam is configured to engage features of the air chamber assembly 254.

The nasal dam 224 as used herein abut a top part of the air chamber assembly 254 and has nares ports 248 that align with air chamber nares ports 242. The nasal dam 224 interfaces with the soft tissue of the nasal base, providing a pressure seal in order to contain airflow between the nasal pharynx and the nasal ventilator system via the nares ports 248/242 and the air chamber 206. The nasal dam may be of a soft Shore A 5-20 durometer material in order to conform to and seal the nasal base from a pressure differential between the air chamber 206 interior and the atmosphere.

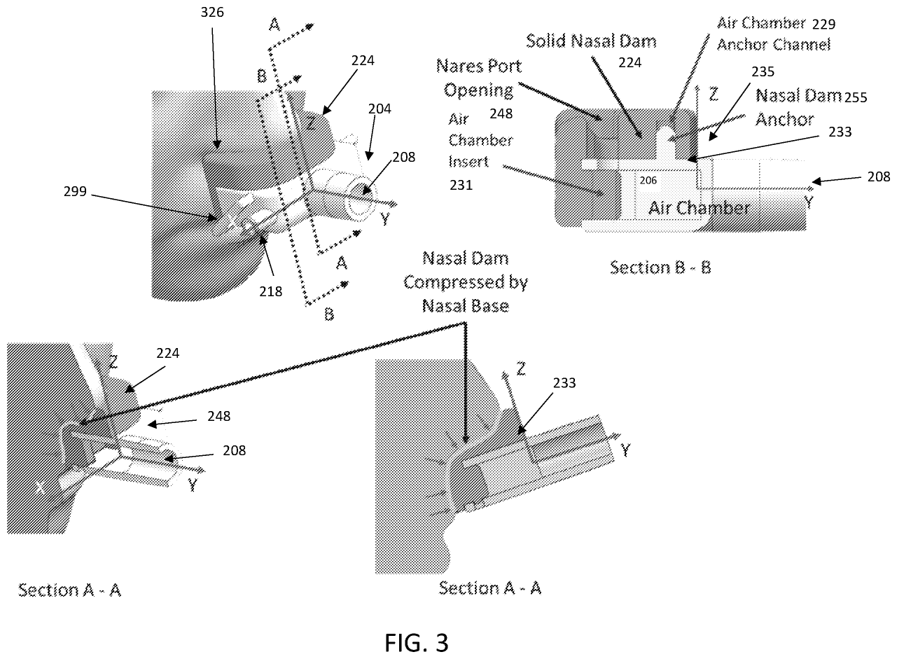

FIG. 3 illustrates interaction of a patient's nasal base 326 and the solid nasal dam 224 of FIG. 2. FIG. 3 includes cross-sectional views of the solid nasal dam 224 with compliance between the patient's nasal base 326 and the top surface 328 of the nasal dam 224. Compliance between a patient's nasal base 326 and a top surface 228 of the nasal dam is involved in achieving a pressure seal between nares of the patient and the air chamber 204 illustrated.

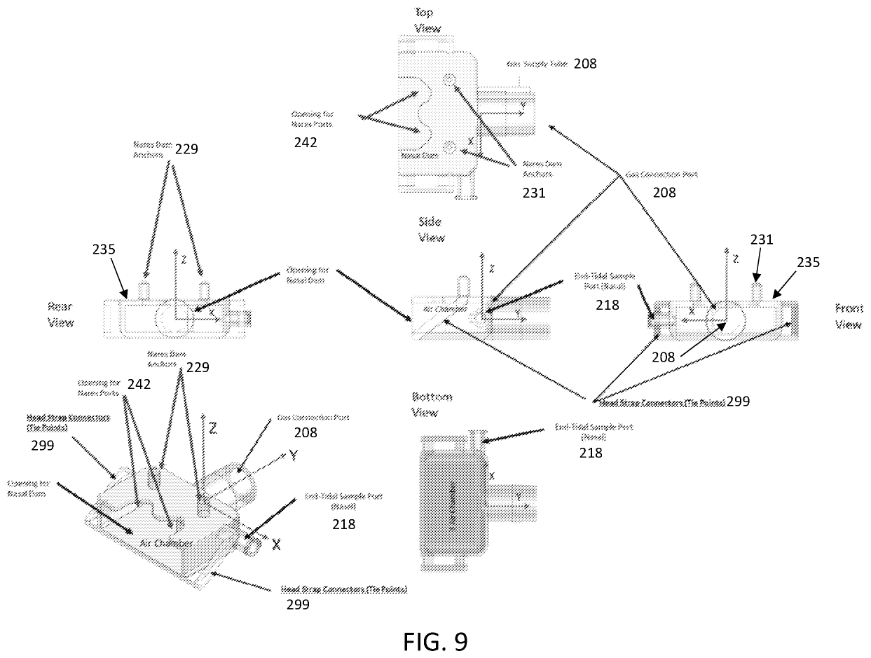

The air chamber assembly 254 includes an air chamber 206, gas and end tidal sample ports (208, 218) in fluid communication with the air chamber 206 and nares ports 242 through an upper wall 260 that correspond to nares ports 248 in the nasal dam 224 that provide fluid communication between the air chamber 206 and the patient's nostrils. In the present example using the disclosed nasal dam 224, the nasal dam 224 is inserted to the rear of the air chamber assembly 254, thus enclosing the rear of the air chamber assembly 254 to provide at least one wall forming the air chamber 206. In addition, as shown in FIG. 3, the nasal dam 224 may include an air chamber insert 231 sized to be received in the rear opening of the air chamber 206 to form a wall of the air chamber. In some embodiments, the back wall of the air chamber 206 may be a rear wall of the air chamber assembly (not shown) or provided by the fit of a portion of the nasal dam 224 into a rear of the air chamber assembly 254. As illustrated in FIG. 9, the air chamber may have a complementary opening for receiving the nasal dam insert 231/531. The air chamber assembly 254 may include head strap or connector tie points 299.

A portion of lower surface 233 of the nasal dam 224 in an X-Y plane abuts an upper portion 235 of the air chamber assembly in the X-Y plane, with the nares ports 248/242 of both the nasal dam and the air chamber assembly aligning when the nasal dam 224 and the air chamber assembly 254 are aligned with one another to allow for fluid communication between the patient's nostrils and the air chamber 206, which in turn is in fluid communication with the gas port 208 and the end tidal sample port 218.

In addition, as shown in FIG. 3, the nasal dam 224 may include an air chamber anchor channel 229, which receives therein a nasal dam anchor 255, which is a protrusion from the upper wall 260 of the air chamber assembly 254, which serves to provide further support for holding the nasal dam 224 in place with respect to air chamber assembly 254.

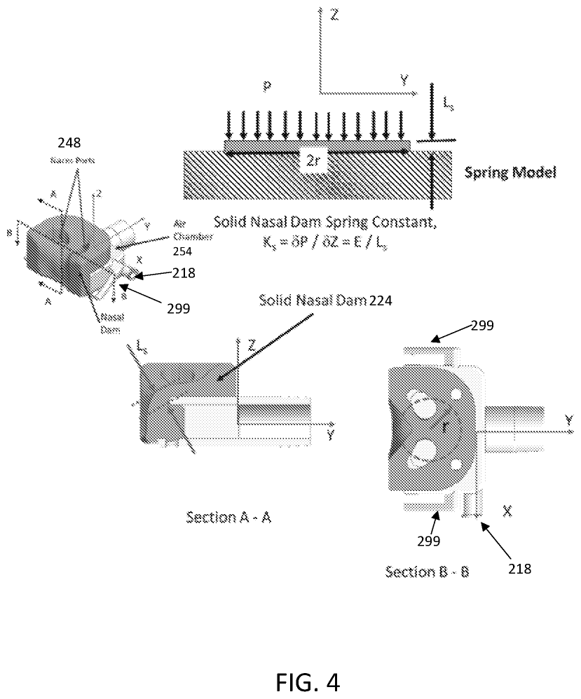

The stiffness of the nasal interface for the solid nasal dam, Ks, as defined by the pressure between the nasal base 326 and nasal dam 224, δP, over an area approximated by a circle of radius r, and resulting in a displacement δZ, as illustrated in FIG. 4 and modeled by Equation (1), below.

Kₛ=δP/δZ=E/Lₛ=7.5×10⁷ N/m³ (1)
Where E=Young's modulus=3×10⁵ N/m² and
   L=Nominal nasal dam thickness≈0.004 m

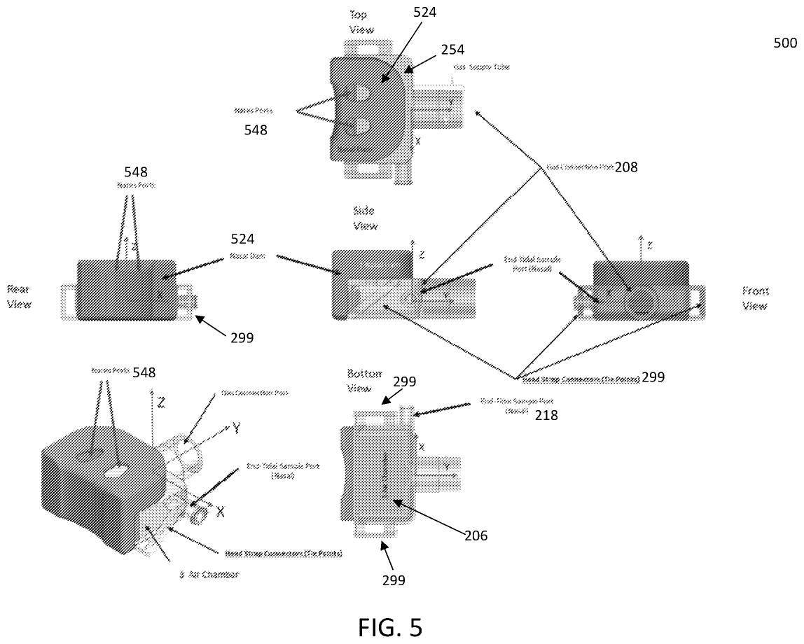

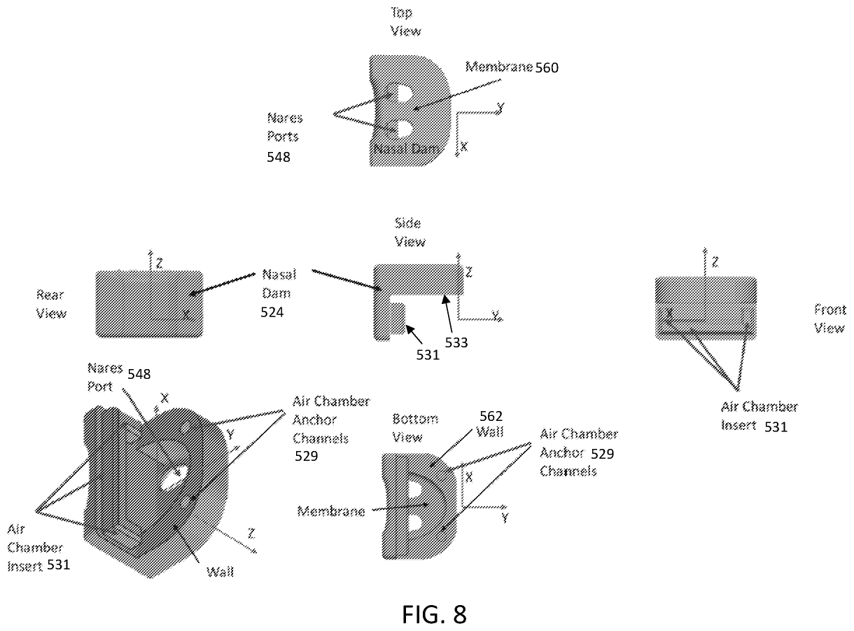

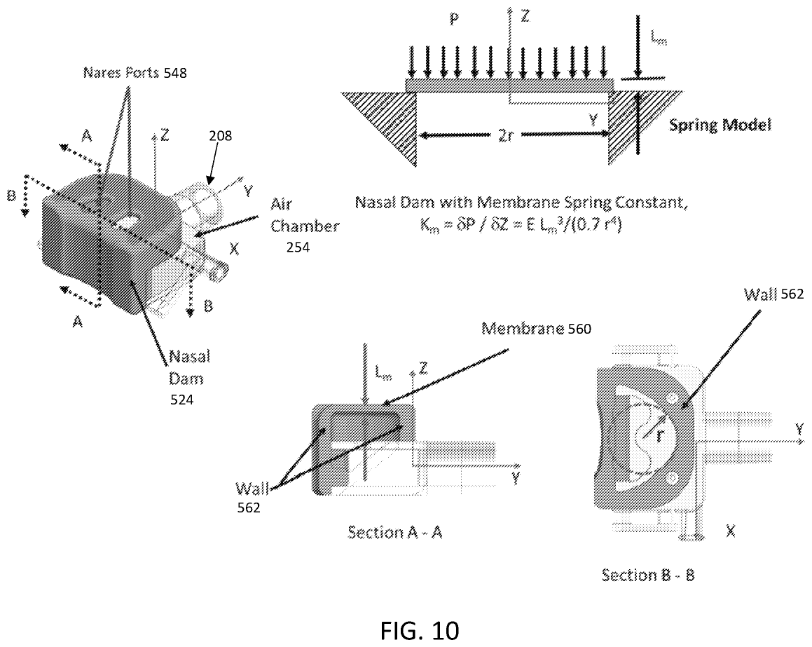

In another embodiment of a nasal respiratory apparatus, a hollow nasal dam may be used to provide comfort and possibly improved sealing against the patient's nasal base. FIGS. 5, 6 and 7 illustrate a nasal respiratory apparatus with a hollow nasal dam 524 and air chamber 254 assembly. FIG. 8 illustrates a hollow nasal dam according to principles described herein. The air chamber 254 may be as previously described or may be a vent, as previously mentioned, or as described further herein. For example, FIG. 9 describes an air chamber that may be used with the hollow nasal dam. FIG. 10 illustrates calculation stiffness of a hollow nasal dam as described herein.

Referring to FIG. 5, a nasal respiratory apparatus 500 with a hollow nasal dam according to principles described herein includes a hollow nasal dam 524. The air chamber assembly 254 includes an air chamber 206, gas and end tidal sample ports (208, 218) in fluid communication with the air chamber 206 and nares ports 242 through an upper wall 260 that correspond to nares ports 548 in the nasal dam 224 that provide fluid communication between the air chamber 206 and the patient's nostrils. In the present example using the disclosed nasal dam 224, the nasal dam 224 is inserted to the rear of the air chamber assembly 254, thus enclosing the rear of the air chamber assembly 254 to provide at least one wall forming the air chamber 206. In addition, as shown in FIG. 3, the nasal dam 224 may include an air chamber insert 231 sized to be received in the rear opening of the air chamber 206 to form a wall of the air chamber. In some embodiments, the back wall of the air chamber 206 may be a rear wall of the air chamber assembly (not shown) or provided by the fit of a portion of the nasal dam 224 into a rear of the air chamber assembly 254. The air chamber assembly 254 may include head strap or connector tie points 299.

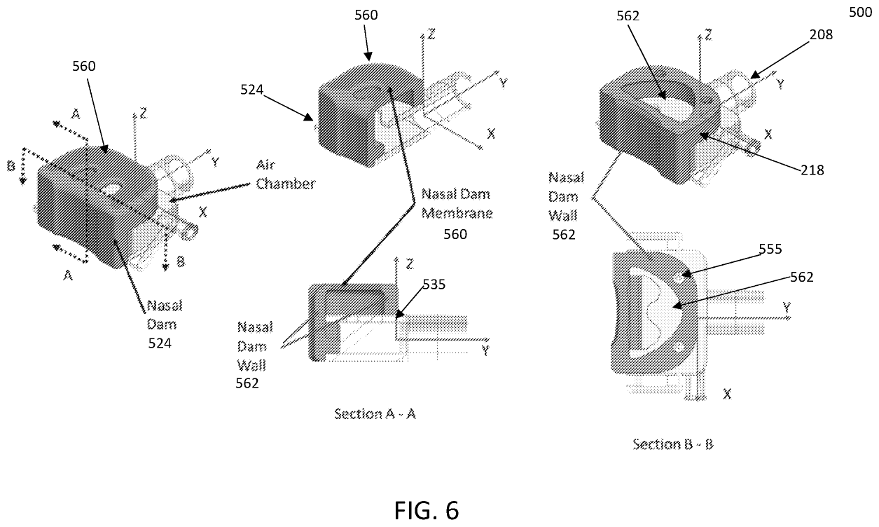

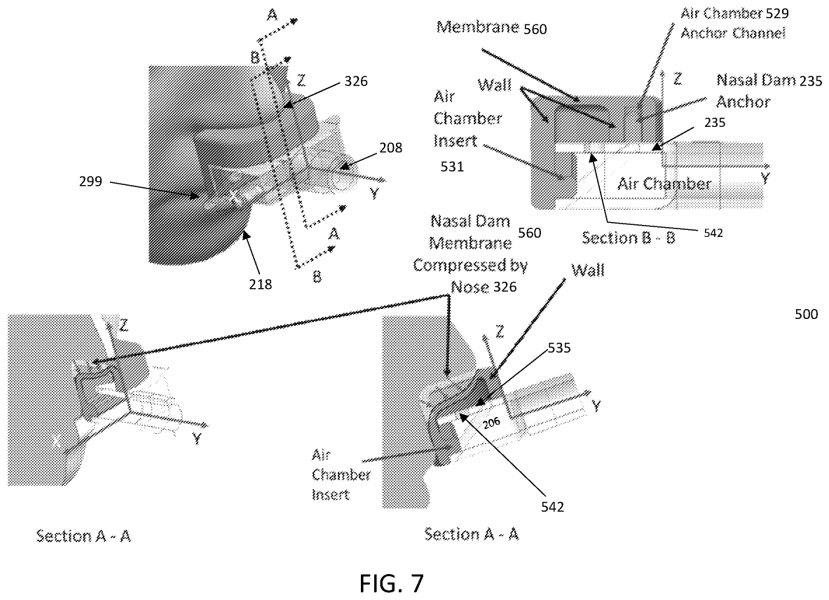

Sectional views of the nasal respiratory assembly 500, as shown in FIGS. 6 and 7, show that the nasal dam 524 is hollow and includes a top membrane 560 in the X-Y plane surrounding at least one nasal dam wall 562 holding up the membrane 560 perpendicular to the X-Y plane of the membrane. That is, the at least one nasal dam wall may be, for example lateral walls 562 of the hollow nasal dam 524, and may be of a pliable material and form a support structure for the top membrane 560 such that the top membrane 560 extends between edges of the lateral walls 562. Compliance between the nasal base 326 of the patient and a top surface 528 of the nasal dam 524 that includes nares ports 548 of the nasal dam 524 (nominally exposed in the X-Y plane on the +Z axis of the nasal dam 524) is involved in achieving a pressure seal between the nares of the patient and the air chamber 206 of the air chamber assembly 254.

Details of the hollow nasal dam 524 are described with reference to FIG. 8. The hollow nasal dam 524 has a membrane 560 supported by the wall to provide an open chamber within the nasal dam 524, where the chamber is in fluid communication with the nares ports 548. The nasal dam 524 may include an air chamber insert 531 such that, when the nasal dam 524 is assembled with the air chamber assembly, the air chamber insert 531 forms a back wall of the air chamber 206 of the air chamber assembly in fluid communication with the gas port and the end tidal CO₂ sampling port, as shown in FIG. 7. As in the previously described solid nasal dam 224, a portion of lower surface 533 of the nasal dam 524 in an X-Y plane abuts an upper portion 235 of the air chamber assembly in the X-Y plane, with the nares ports 548, 542 of both the nasal dam and the air chamber assembly aligning when the nasal dam 524 and the air chamber 254 assembly are aligned with one another to allow for fluid communication between the patient's nostrils and the air chamber 206, which in turn is in fluid communication with the gas port 208 and the end tidal sample port 218. As with the solid nasal dam 224, the hollow nasal dam 524 may attach to the air chamber assembly 254 via anchors 255 extending from the upper portion 235 of the air chamber assembly 254, as illustrated in FIG. 9.

The air chamber assembly 254 mates with the hollow nasal dam as illustrated in FIG. 10. For the hollow nasal dam assembly versus the solid nasal dam assembly, above, it is possible for the region where the nares opening exists to be enlarged and the structure in the X-Y plan adjacent to the nasal dam can be removed, allowing for a less stiff nasal dam-patient interface.

The stiffness of the nasal interface for the hollow nasal dam, Km, as defined by the pressure between the nasal dam 524 and the patient's nasal base, δP, over an area approximated by a circle of radius r, and resulting displacement in δZ has been modeled as a simply-supported circular plate of radius r and thickness Lm, as illustrated in FIG. 10. The spring stiffness Km is modeled by Equation (2), below.

Kₘ=δP/δZ=E Lₘ³/(0.7r⁴)/Lₛ=3.4×10⁵ N/m³ (2)
Where E=Young's modulus=3×10⁵ N/m² and
   L=Nominal nasal dam membrane thickness≈0.002 m
   R=membrane radius≈0.01 m

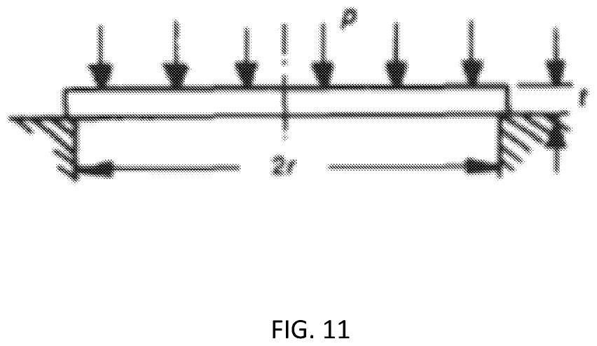

A circular plate for explaining the assumptions and calculations according to Equation (2) is shown in FIG. 11. A circular plate, with uniform load is assumed and shown in FIG. 11.

Symbols used are as follows:
R=radius of circular plate, (m, in)
P=uniform loading, (N/m², lbs/in²)
v=Poisson's ratio (assumed to be 0.3)
E=Young's modulus, (N/m², lbs/in²)
t=plate thickness, (m, in)
σₘ=maximum stress, (N/m², lbs/in²)
yₘ=maximum deflections, (m, in)

Stress at the center of the circular plate of FIG. 11 is given by:

$${σ}_{m} = \frac{3 ( 3 + v ) {pr}^{2}}{8 {t}^{2}} = \frac{1.238 {pr}^{2}}{{t}^{2}}$$

Deflection at the center, with v=0.3 is given by:

$${y}_{m} = \frac{( 5 + v ) {pr}^{4}}{64 ( 1 + v ) D} = \frac{0.696 {pr}^{4}}{{Et}^{3}}$$

Where, D=flexural rigidity=Et³/(12*(1−v²)

Medalist MD10108

| | PSI | Pa |
|---|---|---|
| Elastic Modulus, E | 43.6 | 3.01E+05 |

| | PSI |
|---|---|
| Tensile Stress @ 50% strain | 21.8 |

| | |
|---|---|
| Stiffness | ″E A/L |
| Radius r, m | 0.01 |
| Thickness, Lm, m | 0.002 |
| Stiffness | p/dZ |
| | ELm3(0.7 |
| | r4) |
| | 3.44E+05 |
| Solid Insert | |
| Stiffness | p/dZ |
| | E/Ls |
| | 7.52E+07 |
| Thickness, Ls | 0.004 |

A benefit of the hollow nasal dam design is that the stiffness in the Z direction can be two orders of magnitude smaller, 3.4×10⁵ N/m³ versus 7.5×10⁷ N/m³, for example. As a result, the same displacement in the Z direction required to achieve sealing between the nasal dam and nasal base requires 1/100th of the pressure. This may provide a significant reduction in pressure, which results in better sealing at lower applied pressure from the straps holding the nasal respiratory assembly in place on the patient, for example. Such reduced strap pressure may result in increased patient comfort, providing a reduced risk of pressure ulcers at any patient interface with the nasal respiratory apparatus.

PCT/US2019/068231 may be references for background information regarding a modular nasal dam/air chamber configuration, and relevant portions of that document may be incorporated herein by references as if fully set forth herein for all purposes to the extend allowed by relevant laws.

1. A nasal respiratory assembly, comprising:
a nasal interface comprising at least one opening for fluid communication with the nares of a patient;
an air chamber assembly comprising an air chamber, a gas supply port, an end tidal sample port and at least one opening in fluid communication with the nasal interface,
wherein the nasal interface comprises a pliable material shaped to abut and seal a patient's nasal base such that respiratory gasses pass via the patient's nostrils, the at least one opening, the air chamber, the gas supply port, and the end tidal sample port.

2. The nasal respiratory assembly of claim 1, wherein the nasal interface comprises a solid material having a roughly rectangular cross section and Shore A 5-20 durometers, whereby the at least one opening extends from a surface of the nasal interface through the solid material to an opposite surface of the material and aligns with the at least one opening in fluid communication with the nasal interface.

3. The nasal respiratory device of claim 1, wherein the nasal interface comprises a cavity of material having Shore A 5-20 durometers, and a membrane extending over the cavity, wherein the at least one opening extends through the membrane into the cavity, the cavity further comprising a second opening in a floor of the cavity and in fluid communication with the air chamber via the at least one opening in fluid communication with the nasal interface, whereby a patient's nasal base interface with the membrane for providing a seal.

4. The nasal respiratory assembly of claim 1 wherein the air chamber assembly includes a nasal dam anchor extending from a surface of the air chamber assembly above the air chamber, and nasal interface further comprises an air chamber anchor channel complementary to the nasal dam anchor, whereby insertion of the nasal dam anchor into the air chamber anchor channel provides an interference fit to hold the nasal interface in abutment with the air chamber assembly.

5. The nasal assembly of claim 1, wherein the air chamber or the air chamber assembly includes an open end and the nasal interface includes an air chamber insert complementary to the open end such that insertion of the air chamber insert into the open end of the air chamber provides an interference fit cause the open end to be fluidically sealed.

6. The nasal assembly of claim 3, wherein a spring stiffness of an interface between the membrane and a patient's nasal base is defined by
Kₘ=δP/δZ=E Lₘ³/(0.7r⁴)/Lₛ=3.4×10⁵ N/m³
Where E=Young's modulus=3×10⁵ N/m² and
L=Nominal nasal dam membrane thickness≈0.002 m
R=membrane radius≈0.01 m.

7. The nasal assembly of any one of claim 2, wherein a spring stiffness of an interface between the nasal interface and a patient's nasal base is defined by
Kₛ=δP/δZ=E/Lₛ=7.5×10⁷ N/m³
Where E=Young's modulus=3×10⁵ N/m² and
L=Nominal nasal dam thickness≈0.004 m.
